(19) United States
(12) Patent Application Publication
(10) Pub. No.: US 2016/0074387 A1
(43) Pub. Date: Mar. 17, 2016

(54) Systems and Methods for Attenuating Opioid-Induced Euphoria

(71) Applicant: Purdue Pharma L.P., Stamford, CT (US)

(72) Inventors: Michele Hummel, Marlton, NJ (US); Donald J. Kyle, Yardley, PA (US); Garth Whiteside, Yardley, PA (US)

(21) Appl. No.: 14/853,151

(22) Filed: Sep. 14, 2015

Related U.S. Application Data
(60) Provisional application No. 62/065,393, filed on Oct. 17, 2014.
(60) Provisional application No. 62/049,989, filed on Sep. 12, 2014.

(51) Int. Cl. A61K 31/485

(52) U.S. Cl. CPC A61K 31/485

(57) ABSTRACT

Disclosed in certain embodiments is a method of attenuating or preventing opioid-induced euphoria comprising administering to a patient in need thereof an effective amount of buprenorphine.

This application claims priority to U.S. Provisional Application No. 62/049,989 filed Sep. 12, 2014 and U.S. Provisional Application No. 62/065,393 filed Oct. 17, 2014, both of which are hereby incorporated by reference in their entireties for all purposes.

### FIELD OF THE INVENTION

The invention is directed to systems and methods to attenuate or prevent opioid-induced euphoria.

### BACKGROUND OF THE INVENTION

Endogenous opioids are found throughout the body and are involved in a variety of homeostatic functions and movement control. Receptors that are regulated by endogenous opioids include delta (δ) receptors, kappa (κ) receptors and mu (μ) receptors, all of which are located in the brain and the peripheral nervous system and play a role in analgesia. Of these receptors, the mu (μ) receptors are located in the human gastrointestinal tract on myenteric and submucosal neurons and on immune cells of the lamina propria and play a role in gastrointestinal function.

Exogenous opioids, such as morphine, oxycodone, hydrocodone, buprenorphine and fentanyl, are commonly prescribed to treat both acute and chronic pain, as their action on the opioid receptors can provide effective analgesia. However, with respect to the mu (μ) receptors, the stimulating effect exogenous opioids have on these receptors may also cause euphoria.

Opioid-induced euphoria can be particularly troublesome, as the euphoria produced by the opioid may lead to misuse.

There remains a need in the art for a composition and method to prevent or attenuate opioid-induced euphoria.

All references cited herein are incorporated by reference in their entireties for all purposes.

### OBJECTS AND SUMMARY

It is an object of certain embodiments of the invention to provide methods of attenuating or preventing opioid-induced euphoria.

It is an object of certain embodiments of the invention to provide methods of attenuating or preventing opioid-induced euphoria in a patient on chronic opioid therapy.

It is an object of certain embodiments of the invention to provide methods of attenuating or preventing opioid-induced euphoria in an opioid naive patient.

It is an object of certain embodiments of the invention to provide methods of preventing or reducing misuse and abuse of an opioid by attenuating or preventing opioid-induced euphoria.

It is an object of certain embodiments of the invention to provide methods of attenuating or preventing opioid-induced euphoria resulting from administration of an opioid having an Eₘₐₓ of greater than about 25%.

It is an object of certain embodiments of the invention to provide methods of attenuating or preventing opioid-induced euphoria comprising administering buprenorphine to a patient in need thereof.

It is an object of certain embodiments of the invention to provide pharmaceutical compositions for attenuating or preventing opioid-induced euphoria.

It is an object of certain embodiments of the invention to provide pharmaceutical compositions for attenuating or preventing opioid-induced euphoria in a patient on chronic opioid therapy.

It is an object of certain embodiments of the invention to provide pharmaceutical compositions for attenuating or preventing opioid-induced euphoria in an opioid naive patient.

It is an object of certain embodiments of the invention to provide pharmaceutical compositions for attenuating or preventing opioid-induced euphoria resulting from administration of an opioid having an Eₘₐₓ of greater than about 25%.

It is an object of certain embodiments of the invention to provide pharmaceutical compositions comprising buprenorphine for attenuating or preventing opioid-induced euphoria in a patient in need thereof.

It is an object of certain embodiments of the invention to provide methods of preparing the pharmaceutical compositions disclosed herein for attenuating or preventing opioid-induced euphoria in a patient in need thereof.

It is an object of certain embodiments of the invention to provide kits for attenuating or preventing opioid-induced euphoria in a patient in need thereof.

It is an object of certain embodiments of the invention to provide for the use of buprenorphine in the preparation of a medicament for attenuating or preventing opioid-induced euphoria in a patient in need thereof.

The above objects and others can be achieved by the present invention, which in certain embodiments is directed to a method of attenuating or preventing opioid-induced euphoria comprising administering to a patient in need thereof, an effective amount of buprenorphine to attenuate or prevent opioid-induced euphoria.

In some embodiments, the invention is directed to a method of preventing or treating opioid-induced euphoria comprising administering to a patient in need thereof an effective amount of buprenorphine to prevent, minimize or treat the euphoria induced by the administration of a therapeutically (e.g., analgesically) effective amount of another opioid, e.g., selected from the group consisting of oxycodone, morphine, codeine, oxymorphone, fentanyl, hydrocodone, hydromorphone, tramadol, tapentadol, methadone a pharmaceutically acceptable salt thereof and mixtures thereof. In certain embodiments, the buprenorphine does not cause a substantial decrease, a decrease or an increase in the therapeutic effectiveness (e.g., analgesic effectiveness) of the opioid. In other embodiments, the buprenorphine maintains the therapeutic effectiveness (e.g., analgesic effectiveness) of the opioid. In some embodiments, the buprenorphine is administered in a sub-analgesic amount, e.g., in an amount that would not cause analgesia when administered alone. It is understood that in certain embodiments, the analgesia is decreased but not in an amount that has a negative effect on the analgesia provided to a patient, e.g., the patient does not experience breakthrough pain. A substantial decrease in analgesia would have a negative effect on the analgesia provided to a patient, e.g., the patient experiences breakthrough pain

In certain embodiments, the present invention is directed to a method of attenuating or preventing opioid-induced euphoria comprising administering to a patient on chronic administration of an opioid (other than buprenorphine) having an Eₘₐₓ of greater than about 25%, an effective amount of buprenorphine to attenuate or prevent the opioid-induced euphoria.

In certain embodiments, the present invention is directed to a method of attenuating or preventing opioid-induced euphoria comprising administering to an opioid naive patient an opioid (other than buprenorphine) having an Eₘₐₓ of greater than about 25%, and an effective amount of buprenorphine to attenuate or prevent the opioid-induced euphoria.

In certain embodiments, the present invention is directed to a method of attenuating or preventing an opioid-induced euphoria comprising concurrently administering to a patient in need thereof (i) an effective amount of buprenorphine to attenuate or prevent an opioid-induced euphoria and (ii) another opioid.

In certain embodiments, the present invention is directed to a kit comprising (i) a unit dose of an effective amount of buprenorphine to attenuate or prevent opioid-induced euphoria induced by another opioid and (ii) a unit dose of another opioid in an effective amount to treat pain, diarrhea, cough or anxiety.

In describing the present invention, the following terms are to be used as indicated below. As used herein, the singular forms “a,” “an,” and “the” include plural references unless the context clearly indicates otherwise. Thus, for example, reference to “an opioid” includes a single opioid as well as a mixture of two or more different opioids.

As used herein, the term “therapeutically effective” refers to the amount of drug or the rate of drug administration needed to produce a desired therapeutic result.

As used herein, the term “prophylactically effective” refers to the amount of drug or the rate of drug administration needed to produce a desired preventive result.

The term “patient” means a subject, particularly a human, who has presented a clinical manifestation of a particular symptom or symptoms suggesting the need for treatment, who is treated preventatively or prophylactically for a condition, or who has been diagnosed with a condition to be treated. The term “subject” is inclusive of the definition of the term “patient” and does not exclude individuals who are entirely normal in all respects or with respect to a particular condition.

As used here, the term “patient in need thereof” refers to a patient experiencing opioid-induced euphoria or susceptible to opioid-induced euphoria (e.g., due to past, present or intended administration of an opioid).

“Pharmaceutically acceptable salts” include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate and the like; amino acid salts such as arginate, asparaginate, glutamate and the like; metal salts such as sodium salt, potassium salt, cesium salt and the like; alkaline earth metals such as calcium salt, magnesium salt and the like; and organic amine salts such as triethylamine salt, pyridine salt, picoline salt, ethanolamine salt, triethanolamine salt, discyclohexylamine salt, N,N′-dibenzylethylenediamine salt and the like. The term “buprenorphine” means buprenorphine free base, and all pharmaceutically acceptable salts, complexes, crystalline forms, co-crystals, hydrates, solvates, and mixtures thereof. In certain embodiments, the buprenorphine utilized in the present invention is buprenorphine base or a pharmaceutically acceptable salt thereof (e.g., buprenorphine hydrochloride or buprenorphine levulinate). The term “Cₘₐₓ” denotes the maximum plasma concentration obtained during a dosing interval.

The term “bioavailability” is defined for purposes of the present invention as the relevant extent to which the drug (e.g., oxycodone) is absorbed from a dosage form. Bioavailability is also referred to as AUC (i.e., area under the plasma concentration/time curve).

The term “opioid analgesic” means any material that produces an analgesic effect through modulation of an opioid receptor, whether or not approved by a government agency for that purpose. The term includes all pharmaceutically active forms of the opioid analgesic, including the free base form of the agent, and all pharmaceutically acceptable salts, complexes, crystalline forms, co-crystals, hydrates, solvates, and mixtures thereof, where the form is pharmaceutically active.

The term “opioid-induced euphoria” means a biological reward (e.g., intense feelings of well-being, elation, happiness, ecstasy, excitement and/or joy) experienced by a subject receiving opioid therapy for an intended therapeutic effect or by a subject during misuse of an opioid. Typically, the intended affect is analgesia. The intended effect can also be the treatment of diarrhea, cough, anxiety (e.g., due to shortness of breath) and opioid dependence. The biological reward associated with opioids may be a factor in providing motivation for drug seeking behavior, drug abuse, habituation and/or illicit use of an opioid formulation (e.g., a controlled release oxycodone hydrochloride composition).

The term “peripherally restricted opioid analgesic” means any material that produces an analgesic effect through modulation of a peripheral opioid receptor (whether or not approved by a government agency for that purpose) and does not cross or significantly cross the blood brain barrier. The term includes all pharmaceutically active forms of the peripherally restricted opioid analgesic, including the free base form of the agent, and all pharmaceutically acceptable salts, complexes, crystalline forms, co-crystals, hydrates, solvates, and mixtures thereof, where the form is pharmaceutically active.

The term “concurrently” means that a dose of one agent is administered prior to the end of the dosing interval of another agent. For example, a dose of an opioid analgesic with a 12-hour dosing interval would be concurrently administered with a buprenorphine dose administered within 12 hours of the other opioid analgesic administration.

The term “Eₘₐₓ” means the maximal μ GTP effect elicited by a compound relative (expressed as a %) to the effect elicited by [D-Ala², N-methyl-Phe⁴, Gly-ol⁵]-enkephalin (a/k/a DAMGO), which is a μ agonist standard. Generally, the Eₘₐₓ value measures the efficacy of a compound to treat or prevent pain or diarrhea.

The term “opioid naive” refers to patients who are not receiving opioid analgesics on a daily basis

The term “opioid tolerant” means patients who are chronically receiving opioid analgesics on a daily basis.

The term “first administration” means a single dose at the initiation of therapy to an individual subject, patient, or healthy subject or a subject population, patient population, or healthy subject population.

The term “steady state” means that the amount of the drug reaching the system is approximately the same as the amount of the drug leaving the system. Thus, at “steady-state”, the patient's body eliminates the drug at approximately the same rate that the drug becomes available to the patient's system through absorption into the blood stream.

The term “sub-analgesic” means a dose of a drug (e.g. buprenorphine) that when administered alone does not provide analgesia upon administration to a patient. A sub-analgesic dose does not preclude that the dose can have other therapeutic, prophylactic or pharmacodynamics effects.

### BRIEF DESCRIPTION OF THE DRAWINGS

FIG. 1 is a graphical representation of the results of Example 1.

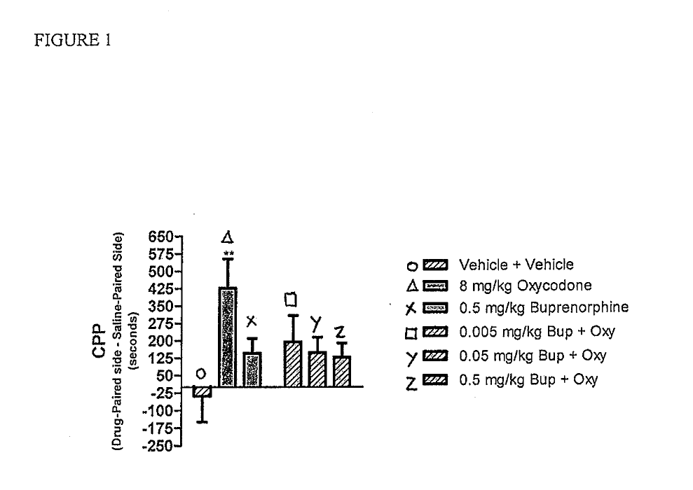

### DETAILED DESCRIPTION

Buprenorphine is commonly used for its analgesic properties and is formulated, e.g., in a transdermal patch (Butrans® buprenorphine transdermal system) to provide 5 mcg/hour, 10 mcg/hour or 20 mcg/hour of buprenorphine. Butrans® is indicated for the management of moderate to severe chronic pain in patients requiring a continuous, around-the-clock opioid analgesic for an extended period of time. The prescribing information states that euphoric mood which is known to occur with opioid treatment is an adverse event (<1%) reported by patients in clinical trials. By virtue of the present invention, buprenorphine can be administered to patients at a dose that will attenuate or prevent euphoria otherwise induced by another opioid.

In certain embodiments, the opioid-induced euphoria can be caused by the administration of an isolated or synthetic opioid that is typically endogenous to the patient (e.g., an endorphin or an enkephalin). In other embodiments, the opioid-induced euphoria can be induced by administration to the patient of another opioid that is exogenous to the patient (e.g., oxycodone, morphine, codeine, oxymorphone, fentanyl, hydrocodone, hydromorphone, tramadol, tapentadol, methadone or a pharmaceutically acceptable salt thereof).

In certain embodiments, the opioid-induced euphoria can be induced by a peripherally restricted opioid, e.g., by administration of a peripherally restricted opioid exogenous to the patient by any suitable route (e.g., parenterally, subcutaneously or intramuscularly).

The peripherally restricted opioid analgesic utilized in the present invention (i) does not cross the blood brain or (ii) does not significantly cross the blood brain barrier (i.e., crosses the blood brain barrier in an amount insufficient to provide a pharmacological effect). The opioid analgesic utilized in the present invention can be peripherally restricted due to, e.g., (i) having an ionic charge (anionic or cationic), (ii) containing a quaternary amine, (iii) molecule size (e.g., proteins and peptides) or (iv) being a p-glycoprotein substrate.

In certain embodiments, the peripherally restricted opioid analgesic is loperamide or a pharmaceutically acceptable salt thereof or frakefamide or a pharmaceutically acceptable salt thereof.

When the peripherally restricted opioid analgesic is loperamide, the agent can be administered subcutaneously, e.g., in an amount from about 0.1 mg/kg to about 10 mg/kg; from about 0.5 mg/kg to about 5 mg/kg, or in an amount of about 1 mg/kg, 2 mg/kg, 3 mg/kg, or 4 mg/kg.

In certain embodiments, the buprenorphine is administered concurrently with another opioid, and the buprenorphine serves to reduce, attenuate, prevent, minimize, inhibit, ameliorate or reverse the opioid-induced euphoria that might otherwise be associated with or caused by the other opioid. Typically, the other opioid is administered in an effective amount to provide an analgesic effect. In other embodiments, the other opioid is administered in an effective amount to treat diarrhea, cough, anxiety (e.g., due to shortness of breath) or opioid dependence. In certain embodiments, the buprenorphine is administered in a sub-analgesic dose, yet still in an effective amount to reduce, attenuate, prevent, minimize, inhibit, ameliorate or reverse the opioid-induced euphoria that might otherwise be associated with or caused by the other opioid.

A patient receiving the buprenorphine therapy of the present invention may be opioid naive. Opioid naive patients may have initiated therapy with the other opioid prior to initiation of the buprenorphine therapy, or they may have initiated therapy with the other opioid concurrently with the initiation of the buprenorphine therapy. In other embodiments, the buprenorphine therapy can be initiated prior to the initiation of therapy with the other opioid so as to provide a prophylactic effect.

Alternatively, a patient receiving the buprenorphine therapy of the present invention may previously have been dosed chronically with another opioid so that he or she is now opioid tolerant.

The buprenorphine therapy of the present invention can be administered after the patient begins to exhibit symptoms of opioid-induced euphoria. Alternatively, the buprenorphine therapy of the present invention can be administered prior to or at the same time as a patient begins treatment with the other opioid in order to reduce or avoid euphoria that might otherwise occur due to administration of the other opioid alone.

In certain embodiments, the other opioid administered before, concurrently with, or after the buprenorphine therapy of the present invention, has an Eₘₐₓ of greater than about 25%, greater than about 40%, greater than about 50%, greater than about 60%, greater than about 70%, greater than about 80%, or greater than about 90%. In certain embodiments, the Eₘₐₓ is from about 25% to about 100%, from about 25% to about 99%, from about 25% to about 95%, from about 25% to about 90%, from about 40% to about 100%, from about 40% to about 99%, from about 40% to about 95%, from about 40% to about 90%, from about 50% to about 100%, from about 50% to about 99%, from about 50% to about 95%, from about 50% to about 90%, from about 60% to about 100%, from about 60% to about 99%, from about 60% to about 95%, from about 60% to about 90%, from about 70% to about 100%, from about 70% to about 99%, from about 70% to about 95%, from about 70% to about 90%, from about 80% to about 100%, from about 80% to about 99%, from about 80% to about 95%, or from about 80% to about 90%

The buprenorphine administered in the present invention can be selected from buprenorphine base, pharmaceutically acceptable salts, solvates, polymorphs, and mixtures thereof. In one embodiment the buprenorphine is administered as buprenorphine hydrochloride. In another embodiment the buprenorphine is administered as buprenorphine levulinate.

The buprenorphine used according to the present invention can be administered by the same route as the other opioid. For example, the buprenorphine and the other opioid can both be administered by the same route selected from the group consisting of oral, transdermal, sublingual, buccal, intranasal, rectal, subcutaneous, intramuscular, intravenous and parenteral.

In alternative embodiments, the buprenorphine used according to the present invention can be administered by a different route than the other opioid. For example, the buprenorphine and the other opioid can be independently administered by different routes selected from the group consisting of oral, transdermal, sublingual, buccal, intranasal, rectal, subcutaneous, intramuscular, intravenous and parenteral.

Non-limiting examples of routes of administration for the present invention include transdermal buprenorphine with the other opioid administered orally; transdermal buprenorphine with the other opioid administered parenterally; transdermal buprenorphine with the other opioid administered intranasally; transdermal buprenorphine with the other opioid administered sublingually; and transdermal buprenorphine with the other opioid administered transdermally.

Other routes of administration of the present invention include sublingual buprenorphine with the other opioid administered orally; sublingual buprenorphine with the other opioid administered parenterally; sublingual buprenorphine with the other opioid administered intranasally; sublingual buprenorphine with the other opioid administered sublingually; and sublingual buprenorphine with the other opioid administered transdermally.

Other routes of administration of the present invention include oral buprenorphine with the other opioid administered orally; oral buprenorphine with the other opioid administered parenterally; oral buprenorphine with the other opioid administered intranasally; oral buprenorphine with the other opioid administered sublingually; and oral buprenorphine with the other opioid administered transdermally.

Other routes of administration of the present invention include parenteral buprenorphine with the other opioid administered orally; parenteral buprenorphine with the other opioid administered parenterally; parenteral buprenorphine with the other opioid administered intranasally; parenteral buprenorphine with the other opioid administered sublingually; and parenteral buprenorphine with the other opioid administered transdermally.

In one embodiment, the buprenorphine is administered in a transdermal system to provide, e.g., a dosing interval of about 24 hours, a dosing interval of about 3 days, or a dosing interval of about 7 days.

The transdermal buprenorphine system can be formulated to administer buprenorphine, e.g., at a rate from about 0.001 mcg/hour to about 50 mcg/hour, from about 0.01 mcg/hour to about 40 mcg/hour, from about 0.05 mcg/hour to about 30 mcg/hour, from about 0.1 mcg/hour to about 20 mcg/hour or from about 0.5 mcg/hour to about 10 mcg/hour. The rate can also be, e.g., from about 0.0001 mcg/hour to about 50 mcg/hour, from about 0.001 mcg/hour to about 40 mcg/hour, from about 0.001 mcg/hour to about 30 mcg/hour, from about 0.001 mcg/hour to about 20 mcg/hour or from about 0.001 mcg/hour to about 10 mcg/hour. The rate can also be, e.g., from about 0.001 mcg/hour to about 50 mcg/hour, from about 0.01 mcg/hour to about 40 mcg/hour, from about 0.01 mcg/hour to about 30 mcg/hour, from about 0.01 mcg/hour to about 20 mcg/hour or from about 0.01 mcg/hour to about 10 mcg/hour

In other embodiments, the transdermal buprenorphine system can be formulated to administer buprenorphine, e.g., at a rate from about 0.001 mcg/hour to about 5 mcg/hour, from about 0.01 mcg/hour to about 4 mcg/hour, from about 0.05 mcg/hour to about 3 mcg/hour, from about 0.1 mcg/hour to about 2 mcg/hour, or from about 0.5 mcg/hour to about 1 mcg/hour. The rate can also be, e.g., from about 0.0001 mcg/hour to about 5 mcg/hour, from about 0.001 mcg/hour to about 4 mcg/hour, from about 0.001 mcg/hour to about 3 mcg/hour, from about 0.001 mcg/hour to about 2 mcg/hour, or from about 0.001 mcg/hour to about 1 mcg/hour. The rate can also be, e.g., from about 0.001 mcg/hour to about 5 mcg/hour, from about 0.01 mcg/hour to about 4 mcg/hour, from about 0.01 mcg/hour to about 3 mcg/hour, from about 0.01 mcg/hour to about 2 mcg/hour, or from about 0.01 mcg/hour to about 1 mcg/hour

In other embodiments, the transdermal buprenorphine system can be formulated to administer buprenorphine, e.g., at a rate of about 50 mcg/hour, about 40 mcg/hour, about 30 mcg/hour, about 20 mcg/hour, about 10 mcg/hour, about 5 mcg/hour, about 4 mcg/hour, about 3 mcg/hour, about 2 mcg/hour, about 1 mcg/hour, about 0.5 mcg/hour, about 0.1 mcg/hour, about 0.05 mcg/hour, about 0.01 mcg/hour, about 0.001 mcg/hour, or about 0.0001 mcg/hour.

In one embodiment, the buprenorphine is administered sublingually. The buprenorphine can be formulated in a sublingual formulation to provide, e.g., a dosing interval of about 0.25 hour, a dosing interval of about 0.5 hour, a dosing interval of about 1 hour, a dosing interval of about 2 hours, a dosing interval of about 3 hours, a dosing interval of about 4 hours, a dosing interval of about 6 hours, a dosing interval of about 8 hours, a dosing interval of about 12 hours, or a dosing interval of about 24 hours.

The sublingual buprenorphine formulation can be formulated to administer buprenorphine, e.g., at a dose of about 0.0001 mg to about 20 mg, from about 0.001 mg to about 10 mg, from about 0.01 mg to about 8 mg, from about .05 mg to about 6 mg, from about 0.1 mg to about 5 mg, from about 0.5 mg to about 4 mg, or from about 1 mg to about 2 mg. The sublingual formulation can also administer a dose, e.g., of from about 0.0001 mg to about 12 mg, from about 0.0001 mg to about 16 mg, from about 0.001 mg to about 8 mg, from about 0.001 mg to about 6 mg, from about 0.001 mg to about 5 mg or from about 0.001 mg to about 4 mg, or from about0 .001 mg to about 2 mg.

In one embodiment, the buprenorphine is administered in an oral dosage form to provide, e.g., a dosing interval of about 4 hours, about 6 hours, about 8 hours, about 12 hours or about 24 hours.

The oral buprenorphine dosage form can be formulated to administer buprenorphine, e.g., at a dose of less than about 500 mg, less than about 400 mg, less than about 350 mg, less than about 300 mg, less than about 250 mg, less than about 200 mg, less than about 150 mg, less than about 100 mg, less than about 90 mg, less than about 80 mg, less than about 70 mg, less than about 60 mg, less than about 50 mg, less than about 40 mg, less than about 30 mg, less than about 20 mg, less than about 10 mg, less than about 9 mg, less than about 8 mg, less than about 7 mg, less than about 6 mg, less than about 5 mg, less than about 4 mg, less than about 3 mg, less than about 2 mg, less than about 1 mg, less than about 0.9 mg, less than about 0.8 mg, less than about 0.7 mg, less than about 0.6 mg, less than about 0.5 mg, less than about 0.4 mg, less than about 0.3 mg, less than about 0.2 mg, less than about 0.1 mg, or less than about 0.01 mg. In certain embodiments, the oral dosage form can administer buprenorphine at a dose of at least about .0001 mg, at least about .001 mg, at least about .01 mg or at least about .1 mg.

In other embodiments, the oral buprenorphine dosage form can be formulated to administer buprenorphine, e.g., at a dose of from about 1 mg to about 500 mg, from about 1 mg to about 400 mg, from about 1 mg to about 350 mg, from about 1 mg to about 300 mg, from about 1 mg to about 250 mg, from about 1 mg to about 200 mg, from about 1 mg to about 150 mg, from about 1 mg to about 100 mg, from about 1 mg to about 90 mg, from about 1 mg to about 80 mg, from about 1 mg to about 70 mg, from about 1 mg to about 60 mg, from about 1 mg to about 50 mg, from about 1 mg to about 40 mg, or from about 1 mg to about 30 mg.

In other embodiments, the oral buprenorphine dosage form can be formulated to administer buprenorphine, e.g., at a dose of from about 30 mg to about 500 mg, from about 30 mg to about 400 mg, from about 30 mg to about 350 mg, from about 30 mg to about 300 mg, from about 30 mg to about 250 mg, from about 30 mg to about 200 mg, from about 30 mg to about 150 mg, from about 30 mg to about 100 mg, from about 30 mg to about 90 mg, from about 30 mg to about 80 mg, from about 30 mg to about 70 mg, from about 30 mg to about 60 mg, from about 30 mg to about 50 mg, or from about 30 mg to about 40 mg.

In other embodiments, the oral buprenorphine dosage form can be formulated to administer buprenorphine, e.g., at a dose of from about 0.0001 mg to about 30 mg, from about 0.001 mg to about 30 mg, from about 0.01 mg to about 30 mg, from about 0.1 mg to about 30 mg, from about 0.2 mg to about 30 mg, from about 0.3 mg to about 30 mg, from about 0.4 mg to about 30 mg, from about 0.5 mg to about 30 mg, from about 0.6 mg to about 30 mg, from about 0.7 mg to about 30 mg, from about 0.8 mg to about 30 mg, from about 0.9 mg to about 30 mg, from about 2 mg to about 30 mg, from about 3 mg to about 30 mg, from about 4 mg to about 30 mg, from about 5 mg to about 30 mg, from about 6 mg to about 30 mg, from about 7 mg to about 30 mg, from about 8 mg to about 30 mg, from about 9 mg to about 30 mg or from about 10 mg to about 30 mg.

In other embodiments, the oral buprenorphine dosage form can be formulated to administer buprenorphine, e.g., at a dose of from about 3 mg to about 500 mg, from about 3 mg to about 400 mg, from about 3 mg to about 350 mg, from about 3 mg to about 300 mg, from about 3 mg to about 250 mg, from about 3 mg to about 200 mg, from about 3 mg to about 150 mg, from about 3 mg to about 100 mg, from about 3 mg to about 90 mg, from about 3 mg to about 80 mg, from about 3 mg to about 70 mg, from about 3 mg to about 60 mg, from about 3 mg to about 50 mg, from about 3 mg to about 40 mg, from about 3 mg to about 30 mg, from about 3 mg to about 20 mg or from about 3 mg to about 10 mg.

In other embodiments, the oral buprenorphine dosage form can be formulated to administer buprenorphine, e.g., at a dose of from about 0.0001 mg to about 3 mg, from about 0.001 mg to about 3 mg, from about 0.01 mg to about 3 mg, from about 0.1 mg to about 3 mg, from about 0.2 mg to about 3 mg, from about 0.3 mg to about 3 mg, from about 0.4 mg to about 3 mg, from about 0.5 mg to about 3 mg, from about 0.6 mg to about 3 mg, from about 0.7 mg to about 3 mg, from about 0.8 mg to about 3 mg, from about 0.9 mg to about 3 mg, from about 1 mg to about 3 mg, or from about 2 mg to about 3 mg.

In certain embodiments, the buprenorphine is administered orally in an amount of from about 0.0001 mg to about 500 mg, from about 0.001 mg to about 500 mg, from about 0.01 mg to about 500 mg, from about 0.1 mg to about 500 mg, from about 0.1 mg to about 400 mg, from about 0.1 mg to about 300 mg, from about 0.1 mg to about 200 mg, from about 0.1 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, or from about 0.1 mg to about 5 mg.

The buprenorphine of the present invention can be administered by any route (e.g., oral or transdermal or subcutaneous) to provide at steady state, e.g., from about .0001 mg/kg to about 1 mg/kg, from about .001 mg/kg to about 1 mg/kg, from about .005 mg/kg to about 0.5 mg/kg or from about .05 mg/kg to about 0.1 mg/kg. In other embodiments, the buprenorphine of the present invention can be administered by any route (e.g., oral) to provide at steady state, e.g., about 1 mg/kg, about 0.5 mg/kg, about 0.1 mg/kg, about .05 mg/kg, about .005 mg/kg, about .001 mg/kg, or about .0001 mg/kg. The buprenorphine can be administered for any suitable time, e.g., for the full duration of therapy with the other opioid or for a fraction of the full duration of therapy with the other opioid.

The buprenorphine of the present invention can be administered by any route (e.g., oral or transdermal or subcutaneous) to provide after first administration or at steady state, a Cₘₐₓ, e.g., from about 0.0001 ng/ml to about 15 ng/ml, from about 0.001 ng/ml to about 15 ng/ml, from about 0.005 ng/ml to about 12 ng/ml, from about 0.05 ng/ml to about 10 ng/ml, from about 0.05 ng/ml to about 1 ng/ml, from about 0.05 ng/ml to about 0.5 ng/ml from about 0.5 ng/ml to about 8 ng/ml, from about 1.0 ng/ml to about 5 ng/ml, or from about 2 ng/ml to about 4 ng/ml.

In other embodiments, the buprenorphine of the present invention can be administered by any route (e.g., oral or transdermal or subcutaneous) to provide after first administration or at steady state, a Cₘₐₓ, e.g., of about 0.0001 ng/ml, about .001 ng/ml, about 0.01 ng/ml, about 0.1 ng/ml, about 1 ng/ml, about 2 ng/ml, about 3 ng/ml, about 4 ng/ml, or about 5 ng/ml.

In other embodiments, the buprenorphine of the present invention can be administered by any route (e.g., oral or transdermal or subcutaneous) to provide after first administration or at steady state, a Cₘₐₓ, e.g., of less than about 5 ng/ml, less than about 4 ng/ml, less than about 3 ng/ml, less than about 2 ng/ml, less than about 1 ng/ml, less than about 0.1 ng/ml, less than about 0.01 ng/ml, less than about 0.001 ng/ml or less than about 0.0001 ng/ml. In certain embodiments, the Cₘₐₓ is, e.g., at least about 0.0001 ng/ml, at least about 0.001 ng/ml, at least about 0.01 ng/ml, or at least about 0.1 ng/ml. In other embodiments, the Cₘₐₓ is, e.g., at least about 0.0001 ng/ml to about 5 ng/ml, at least about 0.001 ng/ml to about 4 ng/ml, at least about 0.01 ng/ml to about 3 ng/ml, or at least about 0.1 ng/ml to about 2 ng/ml. in another embodiment

In other embodiments, the buprenorphine of the present invention can be administered by any route (e.g., oral or transdermal or subcutaneous) to provide after first administration or at steady state, an AUC, e.g., from about 0.001 ng/ml*hr to about 100 ng/ml*hr, from about 0.01 ng/ml*hr to about 100 ng/ml*hr, from about 0.1 ng/ml*hr to about 75 ng/ml*hr, from about 1.0 ng/ml*hr to about 50 ng/ml*hr, from about 5.0 ng/ml*hr to about 40 ng/ml*hr, or from about 10 ng/ml*hr to about 30 ng/ml*hr.

In certain embodiments, the buprenorphine is administered orally and provides attenuation or prevention of opioid-induced euphoria without a circulating plasma level, or with a plasma level below detectable limits.

The steady state or first administration AUC and Cₘₐₓ values disclosed herein may be obtained by any suitable route of administration such as transdermally, sublingually, buccally, orally, subcutaneously, intramuscularly or by a parenteral depot injection. A depot injection of buprenorphine may be administered by implantation (for example, subcutaneously or intramuscularly) or by intramuscular injection. In such formulations, the release of the buprenorphine is controlled by formulation with a suitable polymeric or hydrophobic material (e.g., polylactic glycolic acid), an ion exchange resin, or as a sparingly soluble derivative (e.g., a sparingly soluble salt). Preferably, the depot injection provides a dosing interval from about 1 day to about 3 months, or about 3 days, about 7 days, about 10 days, about 14 days, about 21 days, about one month, about 6 weeks, or about 2 months.

The other opioid can be selected from the group consisting of alfentanil, allylprodine, alphaprodine, anileridine, benzylmorphine, bezitramide, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tapentadol, tilidine, tramadol, pharmaceutically acceptable salts thereof, and mixtures thereof.

In certain embodiments, the other opioid agonist is selected from the group consisting of codeine, fentanyl, hydromorphone, hydrocodone, methadone, oxycodone, dihydrocodeine, dihydromorphine, morphine, tramadol, oxymorphone, pharmaceutically acceptable salts thereof, and mixtures thereof.

In certain embodiments, the other opioid is oxycodone or a pharmaceutically acceptable salt thereof.

In certain embodiments, the other opioid is hydrocodone or a pharmaceutically acceptable salt thereof.

In certain embodiments, the other opioid is hydromorphone or a pharmaceutically acceptable salt thereof.

In certain embodiments, the other opioid is oxymorphone or a pharmaceutically acceptable salt thereof.

In certain embodiments, the other opioid is morphine or a pharmaceutically acceptable salt thereof.

In certain embodiments, the other opioid is fentanyl or a pharmaceutically acceptable salt thereof.

In certain embodiments, the other opioid is methadone or a pharmaceutically acceptable salt thereof.

In certain embodiments, the other opioid is tapentadol or a pharmaceutically acceptable salt thereof.

The other opioid may be formulated in the free base form, or as a pharmaceutically acceptable salt thereof (e.g., a hydrochloride salt, a sulfate salt or a bitartrate salt).

The other opioid can be administered as a transdermal patch, a liquid oral dosage form, or as a solid oral dosage form in either immediate or controlled release form.

The other opioid can be administered in controlled release form with a dosing interval, e.g., of about 8 hours, about 12 hours or about 24 hours. The other opioid can alternatively be administered in immediate release form with a dosing interval, e.g., of about 2 hours, about 4 hours, about 6 hours or about 8 hours. The other opioid, either in controlled release form or immediate release form, can be utilized in the present invention either alone or in combination with a non-opioid analgesic such as a nonsteroidal anti-inflammatory (“NSAID”) (e.g., acetaminophen, aspirin, ibuprofen, naproxen, diclofenac, or a COX-2 inhibitor). Certain combination products can contain in addition to the other opioid, from about 200 mg to about 800 mg acetaminophen (e.g., about 325 mg, about 500 mg or about 650 mg); from about 200 mg to about 800 mg aspirin (e.g., about 325 mg or about 500 mg); or about 200 mg to about 1000 mg ibuprofen (e.g., about 200 mg, about 400 mg, about 600 mg or about 800 mg).

The other opioid in controlled release form can be oxycodone hydrochloride in an amount, e.g., from about 10 mg to about 160 mg per unit dose. In specific embodiments, each unit dose can provide an amount of oxycodone hydrochloride of about 10 mg, about 20 mg, about 30 mg, about 40 mg, about 50 mg, about 60 mg, about 70 mg, about 80 mg, about 100 mg, about 120 mg or about 160 mg. Controlled release oxycodone hydrochloride utilized in the present invention may be Oxycontin® (Oxycodone hydrochloride extended release tablets) commercially available from Purdue Pharma. The oxycodone hydrochloride in immediate release form can be in an amount from about 2.5 mg to about 50 mg, about 2.5 mg, about 4.5 mg; about 4.8355 mg; about 5 mg, about 7.5 mg, about 10 mg, about 15 mg, about 20 mg, or about 30 mg. Immediate release oxycodone hydrochloride utilized in the present invention may be Tylox® (acetaminophen, oxycodone hydrochloride); Roxilox® (acetaminophen, oxycodone hydrochloride); Percocet® (acetaminophen, oxycodone hydrochloride); Oxycet® (acetaminophen, oxycodone hydrochloride); Roxicet® (acetaminophen, oxycodone hydrochloride); Percodan® (aspirin, oxycodone hydrochloride); Oxaydo® (acetaminophen, oxycodone hydrochloride); or Roxicodone® (oxycodone hydrochloride).

The other opioid in controlled release form can be tramadol hydrochloride in an amount, e.g., from about 100 mg to about 300 mg per unit dose. In specific embodiments, each unit dose can provide an amount of tramadol hydrochloride of about 100 mg, about 150 mg, about 200 mg, about 250 mg, or about 300 mg. Tramadol hydrochloride utilized in the present invention may be Conzip® (Tramadol hydrochloride extended release capsules); Ryzolt® (Tramadol hydrochloride extended release tablets); or Ultram ER® (Tramadol hydrochloride extended release capsules). The tramadol hydrochloride in immediate release form can be in an amount from about 2.5 mg to about 100 mg, about 25 mg, about 37.5 mg or about 50 mg. Immediate release tramadol hydrochloride utilized in the present invention may be Ultracet® (acetaminophen, tramadol hydrochloride); or Rybix ODT® (tramadol hydrochloride orally disintegrating tablet).

The other opioid in controlled release form can be oxymorphone hydrochloride in an amount, e.g., from about 5 mg to about 40 mg per unit dose. In specific embodiments, each unit dose can provide an amount of oxymorphone hydrochloride of about 5 mg, about 10 mg, about 15 mg, about 20 mg, about 25 mg, about 30 mg, about 35 mg or about 40 mg. Oxymorphone hydrochloride utilized in the present invention may be Opana ER® (Oxymorphone hydrochloride extended release tablets). The oxymorphone hydrochloride in immediate release form can be in an amount from about 2.5 mg to about 50 mg, about 2.5 mg, about 5 mg; about 10 mg, about 15 mg, about 20 mg, or about 30 mg. Immediate release oxymorphone hydrochloride utilized in the present invention may be Opana® (oxymorphone hydrochloride).

The other opioid in controlled release form can be hydrocodone bitartrate in an amount, e.g., from about 2 mg to about 200 mg per unit dose. In specific embodiments, each unit dose can provide an amount of hydrocodone bitartrate of about 10 mg, about 150 mg, about 20 mg, about 30 mg, about 40 mg, about 60 mg, about 80 mg, about 100 mg or about 120 mg. Controlled release hydrocodone bitartrate utilized in the present invention may be Hysingla® (Hydrocodone bitartrate extended release tablets) commercially available from Purdue Pharma or Zohydro ER® (Hydrocodone bitartrate extended release capsules). The hydrocodone bitartrate in immediate release form can be in an amount from about 2.5 mg to about 20 mg, about 2.5 mg, about 5 mg; about 7.5 mg, about 10 mg, about 12.5 mg, or about 15 mg. Immediate release hydrocodone bitartrate utilized in the present invention may be Vicodin® (acetaminophen, hydrocodone bitartrate); Zydone® (acetaminophen, hydrocodone bitartrate); Anexsia® (acetaminophen, hydrocodone bitartrate); Lortab® (acetaminophen, hydrocodone bitartrate) or Vicoprofen® (ibuprofen, hydrocodone bitartrate).

The other opioid in controlled release form can be morphine sulfate in an amount, e.g., from about 2 mg to about 200 mg per unit dose. In specific embodiments, each unit dose can provide an amount of morphine sulfate of about 10 mg, about 15 mg, about 20 mg, about 30 mg, about 40 mg, about 45 mg, about 50 mg, about 60 mg, about 70 mg, about 75 mg, about 80 mg, about 90 mg, about 100 mg, about 120 mg, about 130 mg, about 150 mg or about 200 mg. Morphine sulfate utilized in the present invention may be Avinza® (Morphine sulfate extended release capsules); Kadian® (Morphine sulfate extended release capsules); or MS Contin® (Morphine sulfate extended release tablets).

The other opioid in controlled release form can be hydromorphone hydrochloride in an amount, e.g., from about 2 mg to about 200 mg per unit dose. In specific embodiments, each unit dose can provide an amount of hydromorphone hydrochloride of about 8 mg, about 12 mg, about 16 mg, about 32 mg, about 64 mg, or about 128 mg, about 20 mg, about 30 mg, about 40 mg, about 60 mg, about 80 mg, about 100 mg or about 120 mg. Hydromorphone hydrochloride utilized in the present invention may be Exalgo® (Hydromorphone hydrochloride extended-release tablets); or Palladone® (Hydromorphone hydrochloride extended-release capsules). The hydromorphone hydrochloride in immediate release form can be in an amount from about 1 mg to about 10 mg, about 2 mg, about 4 mg or about 8 mg. Immediate release hydromorphone hydrochloride utilized in the present invention may be Dilaudid® (hydromorphone hydrochloride oral tablets).

The other opioid in controlled release form can be tapentadol hydrochloride in an amount, e.g., from about 2 mg to about 400 mg per unit dose. In specific embodiments, each unit dose can provide an amount of tapentadol hydrochloride of about 50 mg, about 100 mg, about 150 mg, or about 250 mg. Tapentadol utilized in the present invention may be Nucynta ER® (Tapentadol extended release oral tablets). The tapentadol hydrochloride in immediate release form can be in an amount from about 2 mg to about 150 mg, about 50 mg, about 75 mg or about 100 mg. Immediate release tapentadol hydrochloride utilized in the present invention may be Nucynta® (Tapentadol oral tablets).

The other opioid can be fentanyl disposed in a transdermal system that delivers the fentanyl in an amount, e.g., of about 12.5 mcg/hr; about 25 mcg/hr; about 50 mcg/hr; about 75 mcg/hr or about 100 mcg/hr. Fentanyl utilized in the present invention can be Duragesic® (fentanyl film, extended release).

The other opioid can be methadone, e.g., methadone hydrochloride in an amount, e.g., from about 2.5 mg to about 100 mg per unit dose. In specific embodiments, each unit dose can provide an amount of methadone hydrochloride of about 2.5 mg, about 5 mg, about 10 mg, about 20 mg, about 40 mg or about 50 mg. The dosage form may be an oral solid dosage form (e.g., a tablet or capsule), a solution, a suspension or a parenteral. Methadone utilized in the present invention may be Dolophine® (methadone hydrochloride tablets); Methadose® (methadone hydrochloride tablet); or Diskets® (methadone hydrochloride tablet). The methadone can be a liquid concentrate for oral use (e.g., 10 mg/mL), an injectable solution (e.g., 10 mg/mL), an oral solution (e.g., 10 mg/5 mL or 5 mg/5 mL), a tablet for oral suspension (e.g., 40 mg) or an oral tablet (e.g., 5 mg or 10 mg).

In certain embodiments, the ratio of the daily dose of buprenorphine to the other opioid is, e.g., less than about 1:5 (w/w), less than about 1:10 (w/w), less than about 1:25 (w/w), less than about 1:50 (w/w), less than about 1:75 (w/w), less than about 1:100 (w/w), less than about 1:150 (w/w), less than about 1:200 (w/w), less than about 1:250 (w/w), less than about 1:500 (w/w), less than about 1:600 (w/w), less than about 1:700 (w/w), less than about 1:850 (w/w), less than about 1:1000 (w/w), or less than about 1:10,000 (w/w). In other embodiments, the ratio of the daily dose of buprenorphine to the other opioid is, e.g., at least 1:10,000 (w/w), at least about 1:1000 (w/w), at least about 1:850 (w/w), at least 1:700 (w/w), at least about 1:600 (w/w), or least about 1:500 (w/w). In alternative embodiments, the ratio of the daily dose of buprenorphine to the other opioid is, e.g., from about 1:5 (w/w) to about 1:10,000 (w/w), from about 1:5 (w/w) to about 1:8,000 (w/w), from about 1:5 (w/w) to about 1:5,000 (w/w), from about 1:5 (w/w) to about 1:2,000 (w/w),from about 1:5 (w/w) to about 1:1000 (w/w), from about 1:5 (w/w) to about 1:850 (w/w), from about 1:5 (w/w) to about 1:700 (w/w), from about 1:5 (w/w) to about 1:600 (w/w), from about 1:5 (w/w) to about 1:500 (w/w), from about 1:5 (w/w) to about 1:400 (w/w), from about 1:5 (w/w) to about 1:200 (w/w), from about 1:5 (w/w) to about 1:100 (w/w), from about 1:5 (w/w) to about 1:80 (w/w), from about 1:5 (w/w) to about 1:50 (w/w), or from about 1:5 (w/w) to about 1:25 (w/w).

In certain embodiments, the buprenorphine is administered transdermally at a rate of about 5 mcg/hr or less (e.g., from about .0001 mcg/hr to about 5 mcg/hr, e.g., from about .001 mcg/hr to about 4 mcg/hr, from about .01 mcg/hr to about 3 mcg/hr, or from about .1 mcg/hr to about 2 mcg/hr) concurrently with oral controlled release oxycodone hydrochloride in a unit dose of about 10 mg, about 20 mg, about 30 mg, about 40 mg, about 50 mg, about 60 mg, about 70 mg, about 80 mg, about 100 mg, about 120 mg or about 160 mg. Preferably, the buprenorphine dosing interval is about 3 days or about 7 days and the oxycodone dosing interval is about 12 hours.

In certain embodiments, the buprenorphine is administered transdermally at a rate of about 5 mcg/hr or less (e.g., from about .0001 mcg/hr to about 5 mcg/hr) concurrently with oral controlled release oxymorphone hydrochloride in a unit dose of about 5 mg, about 10 mg, about 15 mg, about 20 mg, about 25 mg, about 30 mg, about 35 mg or about 40 mg. Preferably, the buprenorphine dosing interval is about 3 days or about 7 days, and the oxymorphone dosing interval is about 12 hours.

In certain embodiments, the buprenorphine is administered transdermally at a rate of about 5 mcg/hr or less (e.g., from about .0001 mcg/hr to about 5 mcg/hr) concurrently with oral controlled release hydrocodone bitartrate in a unit dose of about 20 mg, about 30 mg, about 40 mg, about 60 mg, about 80 mg, about 100 mg or about 120 mg. Preferably, the buprenorphine dosing interval is about 3 days or about 7 days, and the hydrocodone dosing interval is about 12 hours or about 24 hours.

In certain embodiments, the buprenorphine is administered transdermally at a rate of about 5 mcg/hr or less (e.g., from about .0001 mcg/hr to about 5 mcg/hr) concurrently with oral controlled release morphine sulfate in a unit dose of about 15 mg, about 30 mg, about 40 mg, about 60 mg, about 80 mg, about 100 mg, about 120 mg or about 200 mg. Preferably, the buprenorphine dosing interval is about 3 days or about 7 days, and the morphine dosing interval is about 12 hours or about 24 hours.

In certain embodiments, the buprenorphine is administered transdermally at a rate of about 5 mcg/hr or less (e.g., from about .0001 mcg/hr to about 5 mcg/hr) concurrently with oral controlled release hydromorphone hydrochloride in a unit dose of about 8 mg, about 12 mg, about 16 mg, about 32 mg, about 64 mg, or about 128 mg. Preferably, the buprenorphine dosing interval is about 3 days or about 7 days, and the hydromorphone dosing interval is about 12 hours.

In certain embodiments, the buprenorphine is administered transdermally at a rate of about 5 mcg/hr or less (e.g., from about .0001 mcg/hr to about 5 mcg/hr) concurrently with transdermally administered fentanyl at a rate of about 12.5 mcg/hr; about 25 mcg/hr; about 50 mcg/hr; about 75 mcg/hr or about 100 mcg/hr. Preferably, the buprenorphine dosing interval is about 3 or 7 days and the fentanyl dosing interval is about 3 or 7 days.

In certain embodiments, the buprenorphine is administered orally concurrently with oral administration of the other opioid. The buprenorphine can be in the same oral dosage form as the other opioid or can be in a separate oral dosage form from the other opioid.

In certain embodiments, the buprenorphine is administered orally in an amount of about 5 mg or less (e.g., from about 0.0001 mg to about 5 mg), about 4 mg or less (e.g., from about 0.0001 mg to about 4 mg), about 2 mg or less (e.g., from about 0.0001 mg to about 2 mg), about 1 mg or less (e.g., from about 0.0001 mg to about 1 mg), about 0.5 mg or less (e.g., from about 0.0001 mg to about 0.5 mg), about 0.25 mg or less (e.g., from about 0.0001 mg to about 0.25 mg) or about 0.1 mg or less (e.g., from about 0.0001 mg to about .01 mg) concurrently with oral controlled release oxycodone hydrochloride in a unit dose of about 10 mg, about 20 mg, about 30 mg, about 40 mg, about 50 mg, about 60 mg, about 70 mg, about 80 mg, about 100 mg, about 120 mg or about 160 mg. Preferably, the buprenorphine dosing interval is about 12 hours or about 24 hours and the oxycodone dosing interval is about 12 hours.

In certain embodiments, the buprenorphine is administered orally in an amount of about 5 mg or less (e.g., from about 0.0001 mg to about 5 mg), about 4 mg or less (e.g., from about 0.0001 mg to about 4 mg), about 2 mg or less (e.g., from about 0.0001 mg to about 2 mg), about 1 mg or less (e.g., from about 0.0001 mg to about 1 mg), about 0.5 mg or less (e.g., from about 0.0001 mg to about 0.5 mg), about 0.25 mg or less (e.g., from about 0.0001 mg to about 0.25 mg) or about 0.1 mg or less (e.g., from about 0.0001 mg to about 0.01 mg) concurrently with oral controlled release oxymorphone hydrochloride in a unit dose of about 5 mg, about 10 mg, about 15 mg, about 20 mg, about 25 mg, about 30 mg, about 35 mg or about 40 mg. Preferably, the buprenorphine dosing interval is about 12 hours or about 24 hours, and the oxymorphone dosing interval is about 12 hours.

In certain embodiments, the buprenorphine is administered orally in an amount of about 5 mg or less (e.g., from about 0.0001 mg to about 5 mg), about 4 mg or less (e.g., from about 0.0001 mg to about 4 mg), about 2 mg or less (e.g., from about 0.0001 mg to about 2 mg), about 1 mg or less (e.g., from about 0.0001 mg to about 1 mg), about 0.5 mg or less (e.g., from about 0.0001 mg to about 0.5 mg), about 0.25 mg or less (e.g., from about 0.0001 mg to about 0.25 mg) or about 0.1 mg or less (e.g., from about 0.0001 mg to about 0.01 mg) concurrently with oral controlled release hydrocodone bitartrate in a unit dose of about 20 mg, about 30 mg, about 40 mg, about 60 mg, about 80 mg, about 100 mg or about 120 mg. Preferably, the buprenorphine dosing interval is about 12 hours or about 24 hours, and the hydrocodone dosing interval is about 12 hours or about 24 hours.

In certain embodiments, the buprenorphine is administered orally in an amount of about 5 mg or less (e.g., from about .0001 mg to about 5 mg), about 4 mg or less (e.g., from about 0.0001 mg to about 4 mg), about 2 mg or less (e.g., from about 0.0001 mg to about 2 mg), about 1 mg or less (e.g., from about 0.0001 mg to about 1 mg), about 0.5 mg or less (e.g., from about 0.0001 mg to about 0.5 mg), about 0.25 mg or less (e.g., from about 0.0001 mg to about 0.25 mg) or about 0.1 mg or less (e.g., from about 0.0001 mg to about .01 mg) concurrently with oral controlled release morphine sulfate in a unit dose of about 15 mg, about 30 mg, about 40 mg, about 60 mg, about 80 mg, about 100 mg, about 120 mg or about 200 mg. Preferably, the buprenorphine dosing interval is about 12 hours or about 24 hours, and the morphine dosing interval is about 12 hours or about 24 hours.

In certain embodiments, the buprenorphine is administered orally in an amount of about 5 mg or less (e.g., from about 0.0001 mg to about 5 mg), about 4 mg or less (e.g., from about 0.0001 mg to about 4 mg), about 2 mg or less (e.g., from about 0.0001 mg to about 2 mg), about 1 mg or less (e.g., from about 0.0001 mg to about 1 mg), about 0.5 mg or less (e.g., from about 0.0001 mg to about 0.5 mg), about 0.25 mg or less (e.g., from about 0.0001 mg to about 0.25 mg) or about 0.1 mg or less (e.g., from about 0.0001 mg to about .01 mg) concurrently with oral controlled release hydromorphone hydrochloride in a unit dose of about 8 mg, about 12 mg, about 16 mg, about 32 mg, about 64 mg, or about 128 mg. Preferably, the buprenorphine dosing interval is about 12 hours or about 24 hours, and the hydromorphone dosing interval is about 12 hours.

In certain embodiments, the buprenorphine is administered orally in an amount of about 5 mg or less (e.g., from about 0.0001 mg to about 5 mg), about 4 mg or less (e.g., from about 0.0001 mg to about 4 mg), about 2 mg or less (e.g., from about 0.0001 mg to about 2 mg), about 1 mg or less (e.g., from about 0.0001 mg to about 1 mg), about 0.5 mg or less (e.g., from about 0.0001 mg to about 0.5 mg), about 0.25 mg or less (e.g., from about 0.0001 mg to about 0.25 mg) or about 0.1 mg or less (e.g., from about 0.0001 mg to about .01 mg) concurrently with transdermally administered fentanyl in an amount of about 12.5 mcg/hr; about 25 mcg/hr; about 50 mcg/hr; about 75 mcg/hr or about 100 mcg/hr. Preferably, the buprenorphine dosing interval is about 12 hours or about 24 hours and the fentanyl dosing interval is about 3 or 7 days.

The buprenorphine and the other opioid can both be formulated to provide (i) an immediate release from the same or different oral dosage forms or (ii) controlled release from the same or different dosage forms.

In alternate embodiments, the buprenorphine can be formulated for immediate release and the other opioid can be formulated for controlled release, from the same or different oral dosage forms.

In further embodiments, the buprenorphine can be formulated for controlled release and the other opioid can be formulated for immediate release, from the same or different oral dosage forms.

Preferably, the oral dosage form containing either the buprenorphine, the other opioid, or both agents, is in the form of a tablet or capsule.

In formulations containing both agents, the buprenorphine and the other opioid can be commingled, blended, or intermixed together in a tablet or capsule.

In a tablet formulation, the core can contain the buprenorphine which is layered with a coating of the other opioid. Alternatively, the core can contain the other opioid which is layered with a coating of the buprenorphine. In other embodiments, the formulation can be in a laminar arrangement such that the buprenorphine and the other opioid are layered in at least a bilayer tablet.

In capsule formulations, the agents can be in the same multiparticulate formulation or in separate multiparticulate formulations that are contained in a pharmaceutically acceptable capsule (e.g., a gelatin capsule). The components of the multiparticulate formulation can be in the form of a core containing the buprenorphine which is layered with a coating of the other opioid. Alternatively, the components of the multiparticulate formulation can be in the form of a core containing the other opioid which is layered with a coating of the buprenorphine. In other embodiments, the capsule can contain a granulation or powder blend containing both the buprenorphine and the other opioid, or separate granulations or powders each containing the buprenorphine or the other opioid.

In oral formulations, the buprenorphine and/or the other opioid can be formulated to provide a delayed release in order to target release at a specific site in the gastro-intestinal tract (e.g., the intestine or the colon). The delayed release can be obtained with an enteric coating on the tablet, multiparticulates, capsule or any other dosage form or component of a dosage form, as appropriate. Enteric materials that can be utilized to provide a delayed release of buprenorphine and/or the other opioid include, e.g., shellac, cellulose acetate phthalate (CAP), polyvinyl acetate phthalate (PVAP), hydroxypropylmethylcellulose phthalate, methacrylic acid ester copolymers and zein.

The invention further encompasses kits that can simplify the administration of buprenorphine concurrently with another opioid in order to prevent or attenuate opioid-induced euphoria. A typical kit of the invention comprises a unit dosage form of buprenorphine and a unit dosage form of another opioid.

In one embodiment, the kit comprises one container holding at least one unit dose of buprenorphine and another container holding at least one unit dose of another opioid. The kit can further comprise a label or printed instructions instructing the use of the buprenorphine to prevent or attenuate opioid-induced euphoria.

Kits of the invention can further comprise a device that is useful for administering the unit dosage forms. Examples of such a device include, but are not limited to, a syringe, a drip bag, a patch, an inhaler, and an enema bag.

In one embodiment, buprenorphine is included in the kit as a transdermal patch, e.g., suitable for administration every 3 or 7 days, along with an amount of unit doses of a controlled or immediate release opioid (e.g., oxycodone hydrochloride, hydrocodone bitartrate or oxymorphone hydrochloride) for an equivalent time period. For example, a kit of the invention can include a 7 day transdermal buprenorphine patch and 14 controlled release oxycodone hydrochloride tablets (to be administered every 12 hours). A kit of the invention can include any combination of a buprenorphine formulation with a formulation the other opioid as disclosed herein. When oral solid dosage forms are included in a kit, the formulations can be contained in a blister package.

The buprenorphine can be in an amount that (i) does not cause a decrease in the analgesic effectiveness of the other opioid, or (ii) does not cause a substantial decrease in the analgesic effectiveness of the other opioid, (iii) provides an increase in analgesia as compared to the administration of the other opioid alone, or maintains the therapeutic effectiveness (e.g., analgesic effectiveness) of the other opioid. The buprenorphine can also be in an amount that (i) causes a decrease in the analgesic effectiveness of the other opioid, or (ii) causes a substantial decrease in the analgesic effectiveness of the other opioid, or (iii) does not provide an increase in analgesia as compared to the administration of the other opioid alone. When the other opioid is utilized for an analgesic effect, it is preferred that the buprenorphine maintains or does not have a negative effect on the analgesic effectiveness of the other opioid.

The concentration of buprenorphine that negatively affects the analgesic efficacy of the concurrently administered other opioid as compared to the concentration of buprenorphine that prevents or attenuates opioid-induced euphoria depends on the identity of the other opioid that is concurrently being administered. Preferably, the window of separation between the concentrations is sufficient such that the buprenorphine effectively prevents or attenuates the opioid-induced euphoria without negatively affecting the analgesic efficacy of the other opioid or with minimal risk of unintentionally obtaining concentrations that negatively affect the analgesic efficacy of the other opioid. For example, oxycodone may have a sufficient window that enables the prevention or attenuation of the opioid-induced euphoria with buprenorphine with a reduced likelihood of the oxycodone having its analgesic effect compromised.

In preferred embodiments, the minimal concentration of buprenorphine that affects the analgesic efficacy of the concurrently administered other opioid is about 1000 times, about 750 times, about 500 times, about 250 times, or about 100 times the concentration of buprenorphine that prevents or attenuates opioid-induced euphoria. In other embodiments, the minimal concentration of buprenorphine that affects the analgesic effectiveness of the concurrently administered other opioid is about 90 times, about 80 times, about 70 times, about 60 times, about 50 times, about 40 times, about 30 times, about 20 times, about 10 times, about 5 times, or about 2 times the minimal concentration of buprenorphine that prevents or attenuates the opioid-induced euphoria. In some embodiments, the minimal concentration of buprenorphine that affects the analgesic efficacy of the concurrently administered other opioid is from about 2 to about 250 times, from about 10 to about 175 times, from about 25 to about 150 times, from about 50 to about 125 times or from about 75 to about 100 times the minimal concentration of buprenorphine that prevents or attenuates the opioid-induced euphoria.

Formulations of Buprenorphine and the Other Opoioid

The buprenorphine and/or the other opioid can be administered as a component of a pharmaceutical composition that comprises a pharmaceutically acceptable carrier or excipient. The buprenorphine and/or the other opioid can be formulated as (i) separate formulations intended for different routes of administration, (ii) separate formulations intended for the same route of administration, or (iii) in the same formulation to be administered together by the same route of administration. The pharmaceutical compositions can be administered by any appropriate route, as determined by the medical practitioner. Methods of administration may include intradermal, intramuscular, intraperitoneal, parenteral, intravenous, subcutaneous, intranasal, epidural, oral, sublingual, buccal, intracerebral, intravaginal, transdermal, transmucosal, rectal, by inhalation, or topical (particularly through the skin).

Pharmaceutical compositions of the invention can take the form of solutions, suspensions, emulsions, tablets, pills, pellets, multi-particulates, capsules, capsules containing liquids, capsules containing powders, capsules containing multi-particulates, lozenges, sustained-release formulations, suppositories, aerosols, sprays, or any other form suitable for use. In one embodiment, the composition is in the form of a capsule (see, e.g., U.S. Pat. No. 5,698,155). Other examples of suitable pharmaceutical compositions and pharmaceutical excipients utilized in the compositions are described in Remington's Pharmaceutical Sciences 1447-1676 (Alfonso R. Gennaro ed., 19th ed. 1995), incorporated herein by reference.

Pharmaceutical compositions of the invention preferably comprise a suitable amount of a pharmaceutically acceptable excipient so as to provide the form for proper administration to the patient. Such a pharmaceutical excipient can be a diluent, suspending agent, solubilizer, binder, disintegrant, buffer, glidant, preservative, coloring agent, lubricant, and the like. The pharmaceutical excipient can be a liquid, such as water or oil, including those of petroleum, animal, vegetable, or synthetic origin, such as peanut oil, soybean oil, mineral oil, sesame oil, and the like. The pharmaceutical excipient can be saline, gum acacia, gelatin, starch paste, talc, keratin, colloidal silica, urea, and the like. In addition, auxiliary, stabilizing, thickening, lubricating, and coloring agents can be used. In one embodiment, the pharmaceutically acceptable excipient is sterile when administered to a patient. Water is a particularly useful excipient when the pharmaceutical composition is administered intravenously. Saline solutions and aqueous dextrose and glycerol solutions can also be employed as liquid excipients, particularly for injectable solutions. Suitable pharmaceutical excipients also include starch, glucose, lactose, sucrose, microcrystalline cellulose, gelatin, malt, rice, flour, chalk, silica gel, sodium stearate, glycerol monostearate, talc, sodium chloride, dried skim milk, glycerol, propylene glycol, water, ethanol, and the like. The compositions of the present invention may also contain minor amounts of wetting or emulsifying agents, or pH buffering agents. Specific examples of pharmaceutically acceptable carriers and excipients that can be used to formulate oral dosage forms are described in the Handbook of Pharmaceutical Excipients, American Pharmaceutical Association (1986).

In certain embodiments, the pharmaceutical compositions are formulated for oral administration. A pharmaceutical composition of the invention to be orally delivered can be in the form of tablets, capsules, gelcaps, caplets, lozenges, aqueous or oily solutions, suspensions, granules, powders, emulsions, syrups, or elixirs, for example. When the buprenorphine and/or the other opioid is incorporated into oral tablets, such tablets can be compressed, tablet triturates, enteric-coated, sugar-coated, film-coated, multiply compressed or multiply layered.

An orally administered pharmaceutical composition can contain one or more additional agents such as, for example, sweetening agents such as fructose, aspartame or saccharin; flavoring agents such as peppermint, oil of wintergreen, or cherry; coloring agents; and preserving agents, and stabilizers, to provide stable, pharmaceutically palatable dosage forms. Techniques and compositions for making solid oral dosage forms are described in Pharmaceutical Dosage Forms: Tablets (Lieberman, Lachman and Schwartz, eds., 2nd ed.) published by Marcel Dekker, Inc. Techniques and compositions for making tablets (compressed and molded), capsules (hard and soft gelatin) and pills are also described in Remington's Pharmaceutical Sciences 1553-1593 (Arthur Osol, ed., 16.sup.th ed., Mack Publishing, Easton, Pa. 1980). Liquid oral dosage forms include aqueous and non-aqueous solutions, emulsions, suspensions, and solutions and/or suspensions reconstituted from non-effervescent granules, optionally containing one or more suitable solvents, preservatives, emulsifying agents, suspending agents, diluents, sweeteners, coloring agents, flavoring agents, and the like. Techniques and compositions for making liquid oral dosage forms are described in Pharmaceutical Dosage Forms: Disperse Systems, (Lieberman, Rieger and Banker, eds.) published by Marcel Dekker, Inc.

When the buprenorphine and/or the other opioid is formulated for parenteral administration by injection (e.g., continuous infusion or bolus injection), the formulation can be in the form of a suspension, solution, or emulsion in an oily or aqueous vehicle, and such formulations can further comprise pharmaceutically necessary additives such as one or more stabilizing agents, suspending agents, dispersing agents, and the like. When the buprenorphine and/or the other opioid is to be injected parenterally, it can be, e.g., in the form of an isotonic sterile solution. The buprenorphine and/or the other opioid can also be in the form of a powder for reconstitution as an injectable formulation.

In certain embodiments, the buprenorphine and/or the other opioid is formulated into a pharmaceutical composition for intravenous administration. Typically, such compositions comprise sterile isotonic aqueous buffer. Where necessary, the compositions can also include a solubilizing agent. A pharmaceutical composition for intravenous administration can optionally include a local anesthetic such as benzocaine or prilocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the buprenorphine and/or the other opioid is to be administered by infusion, it can be dispensed, for example, with an infusion bottle containing sterile pharmaceutical grade water or saline. When the buprenorphine and/or the other opioid is administered by injection, an ampule of sterile water for injection or saline can be provided so that the ingredients can be mixed prior to administration.

When the buprenorphine and/or the other opioid is to be administered by inhalation, it can be formulated into a dry aerosol, or an aqueous or partially aqueous solution.

In another embodiment, the buprenorphine and/or the other opioid can be delivered in a vesicle, in particular a liposome (see Langer, Science 249:1527-1533 (1990); and Treat et al., Liposomes in the Therapy of Infectious Disease and Cancer 317-327 and 353-365 (1989)).

In certain embodiments, the buprenorphine and/or the other opioid can be delivered in an immediate release form. In other embodiments, the buprenorphine and/or the other opioid can be delivered in a controlled-release system or sustained-release system. Controlled- or sustained-release pharmaceutical compositions can have a common goal of improving drug therapy over the results achieved by their non-controlled or non-sustained-release counterparts. Advantages of controlled- or sustained-release compositions include extended activity of the drug, reduced dosage frequency, and increased compliance. In addition, controlled- or sustained-release compositions can favorably affect the time of onset of action or other characteristics, such as blood levels of the buprenorphine and/or the other opioid, and can thus reduce the occurrence of adverse side effects.

Controlled- or sustained-release compositions can initially release an amount of the buprenorphine and/or the other opioid that promptly produces the desired therapeutic or prophylactic effect, and gradually and continually release other amounts of the buprenorphine and/or the other opioid to maintain a level of therapeutic or prophylactic effect over an extended period of time. To maintain a constant level of the buprenorphine and/or the other opioid in the body, the pharmaceutical composition can release the active(s) from the dosage form at a rate that will replace the amount of active(s) being metabolized and excreted from the body. Controlled or sustained release of an active ingredient can be triggered by any of various conditions, including but not limited to, changes in pH, changes in temperature, concentration or availability of enzymes, concentration or availability of water, or other physiological conditions or compounds.

Controlled-release and sustained-release means for use according to the present invention may be modified from those known in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, each of which is incorporated herein by reference. Such dosage forms can be used to provide controlled- or sustained-release of one or both of the active ingredients using, for example, hydropropylmethyl cellulose, other polymer matrices, gels, permeable membranes, osmotic systems, multilayer coatings, microparticles, multiparticulates, liposomes, microspheres, or a combination thereof to provide the desired release profile in varying proportions. Suitable controlled- or sustained-release formulations known in the art, including those described herein, can be readily selected for use with the active ingredients of the invention in view of this disclosure. See also Goodson, “Dental Applications” (pp. 115-138) in Medical Applications of Controlled Release, Vol. 2, Applications and Evaluation, R. S. Langer and D. L. Wise eds., CRC Press (1984). Other controlled- or sustained-release systems that are discussed in the review by Langer, Science 249:1527-1533 (1990) can be selected for use according to the present invention. In one embodiment, a pump can be used (Langer, Science 249:1527-1533 (1990); Sefton, CRC Crit. Ref Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61 (1983); Levy et al., Science 228:190 (1985); During et al., Ann. Neurol. 25:351 (1989); and Howard et al., J. Neurosurg. 71:105 (1989)).

When in tablet or pill form, a pharmaceutical composition of the invention can be coated to delay disintegration and absorption in the gastrointestinal tract thereby providing targeted release to a particular portion of the GI tract, or providing a sustained action over an extended period of time. Selectively permeable membranes surrounding an osmotically active driving compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions preferably include standard excipients of pharmaceutical grade selected, for example, from mannitol, lactose, starch, magnesium stearate, sodium saccharin, cellulose, and magnesium carbonate, among others.

Controlled release oral dosage forms according to the present invention may also be prepared as osmotic dosage forms. The osmotic dosage forms preferably include a bilayer core comprising a drug layer (containing the buprenorphine and/or the other opioid) and a delivery or push layer, wherein the bilayer core is surrounded by a semipermeable wall and optionally having at least one passageway disposed therein.

The expression “passageway” as used for the purpose of this invention, includes an aperture, orifice, bore, pore, porous element, fiber, capillary tube, porous overlay, porous insert, microporous member, or porous composition through any of which the buprenorphine and/or the other opioid can diffuse, migrate or be pumped through. The passageway can also include a compound that erodes or is leached from the wall in the fluid environment of use to produce at least one passageway. Representative compounds for forming a passageway include erodible poly(glycolic) acid or poly(lactic) acid in the wall; a gelatinous filament; a water-removable poly(vinyl alcohol); and leachable compounds such as fluid-removable pore-forming polysaccharides, acids, salts or oxides. Examples of leachable compounds include sorbitol, sucrose, lactose, maltose, or fructose. The passageway can have any shape, such as round, triangular, square and elliptical, for assisting in the controlled release of the buprenorphine and/or the other opioid from the dosage form. The dosage form can be manufactured with one or more passageways in spaced-apart relation on one or more surfaces of the dosage form. A passageway and equipment for forming a passageway are described in U.S. Pat. Nos. 3,845,770; 3,916,899; 4,063,064 and 4,088,864. Passageways prepared by leaching are described in U.S. Pat. Nos. 4,200,098 and 4,285,987.

In certain embodiments the drug layer may comprise at least one polymer hydrogel. Examples of polymer hydrogels include but are not limited to a maltodextrin polymer; a poly(alkylene oxide) such as a poly(ethylene oxide) and a poly(propylene oxide); an alkali carboxyalkylcellulose, wherein the alkali is sodium or potassium and the alkyl is methyl, ethyl, propyl, or butyl; and a copolymer of ethylene-acrylic acid, including methacrylic and ethacrylic acid.

In certain embodiments of the present invention, the delivery or push layer comprises an osmopolymer. Examples of an osmopolymer include but are not limited to a member selected from the group consisting of a polyalkylene oxide and a carboxyalkylcellulose. The polyalkylene oxide may be a member selected from the group consisting of polymethylene oxide, polyethylene oxide and polypropylene oxide. The carboxyalkylcellulose may be a member selected from the group consisting of alkali carboxyalkylcellulose, sodium carboxymethylcellulose, potassium carboxymethylcellulose, sodium carboxyethylcellulose, lithium carboxymethylcellulose, sodium carboxyethylcellulose, carboxyalkylhydroxyalkylcellulose, carboxymethylhydroxyethylcellulose, carboxyethylhydroxyethylcellulose and carboxymethylhydroxypropylcellulose. The osmopolymers used for the displacement layer exhibit an osmotic pressure gradient across the semipermeable wall. The osmopolymers imbibe fluid into the dosage form, thereby swelling and expanding as an osmotic hydrogel, whereby they push the contents of the drug layer from the osmotic dosage form.

The push layer may also include one or more osmotically effective compounds that imbibe an environmental fluid, for example, from the gastrointestinal tract, into the dosage form to contribute to the delivery kinetics of the displacement layer. Examples of osmotically effective compounds comprise a member selected from the group consisting of osmotic salts and osmotic carbohydrates. Examples of specific osmagents include but are not limited to sodium chloride, potassium chloride, magnesium sulfate, lithium phosphate, lithium chloride, sodium phosphate, potassium sulfate, sodium sulfate, potassium phosphate, glucose, fructose and maltose.

The push layer may optionally include a hydroxypropylalkylcellulose such as hydroxypropylmethylcellulose, hydroxypropylethylcellulose, hydroxypropyl isopropyl cellulose, hydroxypropylbutylcellulose, and hydroxypropylpentylcellulose.

In certain alternative embodiments, the dosage form comprises a substantially homogenous core comprising the buprenorphine and/or the other opioid, a pharmaceutically acceptable polymer (e.g., polyethylene oxide) and optional excipients such as disintegrants and absorption enhancers. The substantially homogenous core is surrounded by a semipermeable wall having a passageway (as defined above) for the release of the buprenorphine and/or the other opioid. Such an embodiment would not require a push layer.

In certain embodiments, the semipermeable wall comprises a member selected from the group consisting of a cellulose ester polymer, a cellulose ether polymer and a cellulose ester-ether polymer. Representative wall polymers comprise a member selected from the group consisting of cellulose acylate, cellulose diacylate, cellulose triacylate, cellulose acetate, cellulose diacetate, cellulose triacetate, mono-, di- and tricellulose alkenylates, and mono-, di- and tricellulose alkinylates.

With osmotic systems, the buprenorphine or the other opioid can be formulated for controlled release and the other agent can be formulated for immediate release, e.g., by coating onto the semipermeable wall.

Pharmaceutical compositions of the invention include single unit dosage forms suitable for oral administration such as, but not limited to, tablets, capsules, gelcaps, and caplets, which may be adapted for controlled or immediate release.

In certain embodiments, both the buprenorphine and the other opioid can be included in the same dosage form. For example, the buprenorphine and the other opioid can both be included in a transdermal dosage form such that each agent is administered according to the desired rate. In certain embodiments, the two agents can be segregated from each other in a dual reservoir system.

Transdermal Dosage Forms

In certain embodiments, wherein the buprenorphine is administered in a transdermal device, the formulation can, e.g., be a transdermal patch, a transdermal plaster, a transdermal disc or an iontophoretic transdermal device.

In certain embodiments, transdermal dosage forms of buprenorphine that can be utilized in the present invention are described in WO2008/061625; WO2012/163655; WO2013/088254; WO2014/090921; WO2014/105480; WO2014/195352 and US RE 41,571.

Transdermal dosage forms used in accordance with the invention can include a backing layer made of a pharmaceutically acceptable material which is impermeable to the buprenorphine. The backing layer can serve as a protective cover for the buprenorphine and may also provide a support function. Examples of materials suitable for making the backing layer are films of high and low density polyethylene, polypropylene, polyvinylchloride, polyurethane, polyesters such as poly(ethylene phthalate), metal foils, metal foil laminates of suitable polymer films, textile fabrics, and the like. The backing layer can be any appropriate thickness which will provide the desired protective and support functions. A suitable thickness can be, e.g., from about 10 microns to about 200 microns.

In certain embodiments, the transdermal dosage forms used in accordance with the invention contain a biologically acceptable polymer matrix layer. Generally, the polymers used to form the polymer matrix layer are capable of allowing the buprenorphine to pass through at a controlled rate. A non-limiting list of exemplary materials for inclusion in the polymer matrix includes polyethylene, polypropylene, ethylene/propylene copolymers, ethylene/ethylacrylate copolymers, ethylenevinyl acetate copolymers, silicones, natural or synthetic rubber, polyacrylic esters and copolymers thereof, polyurethanes, polyisobutylene, chlorinated polyethylene, polyvinylchloride, vinyl chloride-vinyl acetate copolymer, polymethacrylates, polyvinylidene chloride, poly(ethylene terephthalate), ethylene-vinyl alcohol copolymer, ethylene-vinyloxyethanol copolymer, silicones, silicone copolymers such as polysiloxane-polymethacrylate copolymers, cellulose polymers (e.g., ethyl cellulose, and cellulose esters), polycarbonates, polytetrafluoroethylene and mixtures thereof.

The polymer matrix layer may optionally include a pharmaceutically acceptable cross-linking agent such as, e.g., tetrapropoxy silane.

In certain embodiments, the transdermal delivery systems used in accordance with the methods of the present invention include an adhesive layer to affix the dosage form to the skin of the patient for a desired period of administration, e.g., about 1 day, about 2 days, about 3 days, about 4 days, about 5 days, about 6 days, or about 7 days. If the adhesive layer of the dosage form fails to provide adhesion for the desired period of time, it is possible to maintain contact between the dosage form with the skin, e.g., by affixing the dosage form to the skin of the patient with an adhesive tape.

The adhesive layer may include an adhesive such as polyacrylic adhesive polymers, acrylate copolymers (e.g., polyacrylate) and polyisobutylene adhesive polymers.

The transdermal dosage forms which can be used in accordance with the present invention may optionally include a permeation enhancing agent. Permeation enhancing agents are compounds which promote penetration and/or absorption of the buprenorphine into the blood stream of the patient. A non-limiting list of permeation enhancing agents includes polyethylene glycols, surfactants, and the like.

In one embodiment, the transdermal dosage form which may be used in accordance with the present invention includes a non-permeable backing layer comprising, e.g., a polyester; an adhesive layer comprising, e.g., a polyacrylate; and a matrix containing the buprenorphine and other excipients such as softeners, permeability enhancers, viscosity agents and the like.

The buprenorphine may be included in the device in a drug reservoir, drug matrix or drug/adhesive layer. Preferably, the active agent is buprenorphine or a pharmaceutically acceptable salt thereof.

Certain preferred transdermal delivery systems also include a softening agent. Suitable softening agents include higher alcohols such as dodecanol, undecanol, octanol, esters of carboxylic acids, diesters of dicarboxylic acids and triglycerides. Further examples of suitable softeners are multivalent alcohols such as levulinic acid, caprylic acids, glycerol and 1,2-propanediol, which can also be etherified by a polyethylene glycol.

A buprenorphine solvent may also be included in the transdermal delivery systems of the present invention. A non-limiting list of suitable solvents includes those with at least one acidic group such as monoesters of dicarboxylic acids (e.g., monomethylglutarate and monomethyladipate).

In certain embodiments, the transdermal dosage form includes a removable protective layer. The removable protective layer is removed prior to application, and may comprise the materials used for the production of the backing layer disclosed above provided that they are rendered removable, e.g., by silicone treatment. Other removable protective layers include polytetra-fluoroethylene, treated paper, allophane, polyvinyl chloride, and the like. Generally, the removable protective layer is in contact with the adhesive layer and provides a convenient means of maintaining the integrity of the adhesive layer until the desired time of application.

The transdermal system utilized in the present invention is used by adhering the transdermal system to a dermal surface of a patient. The dermal surface should be clean and unbroken. In certain embodiments, the transdermal system will be sufficiently adhesive to remain adhered to the patient's skin during normal everyday activities and for an adequate period of time. In other embodiments, it may be necessary to further secure the transdermal system to the patient, e.g., by wrapping tape or a medical bandage around the area to which the transdermal system has been applied.

In some embodiments, the transdermal system can be cut or otherwise separated into two or more separate pieces to adjust the amount of buprenorphine that will be delivered to the patient. For example, the transdermal system may include perforations or lines along which to cut for dividing the transdermal system into multiple doses.

Mucosal Tablets and Films

In certain embodiments, the buprenorphine can be formulated for application to the mucosal tissue. Such a formulation can be a tablet, film or spray adapted for lingual (i.e., to be placed onto the tongue), sublingual (i.e., to be placed under the tongue), buccal (i.e., to be applied to the cheek), or gingival (i.e., to be applied to the gums) administration. One benefit of such administration is the avoidance or reduction of first pass metabolism associated with oral administration.

Sublingual, lingual, buccal and gingival tablets and films are formulated to disintegrate rapidly in the mouth to provide absorption of the buprenorphine in the oral cavity in a relatively short period of time. Such forms may contain soluble excipients such as lactose, mannitol, dextrose, sucrose or mixtures thereof. Such forms may also contain granulating and disintegrating agents such as starch, silicon dioxide, or sodium starch glycolate, binding agents such as povidone or hydroxypropyl-methyl cellulose and lubricating agents such as magnesium stearate. Such forms may also comprise a bioerodible polymeric carrier that optionally may also serve to adhere the dosage form to the sublingual, lingual, buccal, or gingival mucosa.

In some embodiments, the buprenorphine can be formulated as a gel in the form of a film or strip. The film should be capable of disintegrating quickly, e.g., in about 0.5 second to 120 seconds from contact with a surface in the oral cavity. In certain embodiments, the film is capable of disintegrating within about 0.5 second to about 60 seconds, or in less than about 5 seconds, or in less than about 10 seconds, or in less than about 15 seconds, or in less than about 20 seconds, or in less than about 30 seconds, or in less than about 45 seconds.

The film may comprise hydrophilic (water-soluble and water-swellable) polymers that adhere to a wet surface in the oral cavity. Polymeric carriers may be selected from acrylic acid polymers, hydrolyzed polyvinylalcohols, polyethylene oxides, polyacrylates, vinyl polymers, polyvinylpyrrolidones, dextrans, guar gums, pectins; starches, and cellulosic polymers, among others.

Mucosal tablets or films can also include a permeation enhancer to increase the rate at which the buprenorphine permeates through the mucosal tissue to which it is applied, e.g., the buccal, lingual, gingival, or sublingual mucosa. Permeation enhancers may be selected from dimethylsulfoxide (“DMSO”), dimethyl formamide (“DMF”), N,N-dimethylacetamide (“DMA”), decylmethylsulfoxide (“C₁₀MSO”), polyethylene glycol monolaurate (“PEGML”), glycerol monolaurate, lecithin, 1-substituted azacycloheptan-2-ones, alcohols, and surfactants, among others.

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### Example 1

A dose of buprenorphine was tested for the attenuation of opioid-induced euphoria in an animal model using rats and was a modification of the technique described in “Hummel M, Lu P, Cummons T A, Whiteside G T. The persistence of a long-term negative affective state following the induction of either acute or chronic pain. Pain 140:436-445, 2008.” The modified technique is described in detail below.

Apparatus-All conditioning and testing occurred in Plexiglas chambers purchased from Med Associates Inc. (St. Albans, Vermont; Med-CPP-RS). Each chamber consisted of two equal-sized compartments (11×8.25×8.4) separated by a neutral grey compartment (6×8.25×8.4). The compartments were separated by removable partition guillotine-like doors which permitted both restricted and unrestricted access. The two larger compartments which occupy positions to the left and right of the central compartment had different colored walls and distinct flooring. The compartment to the left of the central area had white walls and grid rod flooring, whereas the compartment on the right had black walls and mesh-style flooring.

Materials-The vehicle used was 25% HPBCD (2-Hydroxypropyl)-β-cyclodextrin; Sigma-Aldrich). It was administered in a volume of 2 ml/kg. The buprenorphine was administered in the base form and was formulated in 25% HPBCD. The oxycodone was administered as the hydrochloride salt form and was dissolved in saline. The subcutaneous volume for each was 2 ml/kg.

For this Example, a 6 day unbiased, counterbalanced paradigm was employed. Innate preference was tested on day 1 (preconditioning day or baseline). On this day, naïve animals (rats) had free access to all three compartments for 30 minutes. The time spent in each chamber was automatically recorded. For conditioning sessions (days 2-7), animals were randomly assigned to compartment pairings for thirty minutes. During the first conditioning session (day 2), animals were randomly divided. Half of the animals were confined to the white compartment paired with either vehicle plus vehicle, buprenorphine plus oxycodone, buprenorphine or oxycodone injection while the other half were confined to the alternate black chamber and were similarly paired. Control animals received vehicle plus vehicle during each pairing. This conditioning procedure was replicated over five more days (thirty minute sessions for 6 total conditioning days). On day 8, preference was assessed. Each animal was allowed to roam freely about all three compartments for 30 minutes. The removable doors remained opened. The time spent in each compartment was automatically recorded. Conditioned Place Preference (“CPP”) (seconds) is represented as the difference between the time spent in the drug-paired and vehicle-paired chambers on the test day and is indicative of the rewarding properties associated with the compound(s) studied.

As shown in the results, buprenorphine reduces oxycodone-induced reward as (i) oxycodone alone produces a statistically significant CPP as compared to vehicle treated controls, (ii) buprenorphine co-administered with oxycodone does not produce a statistically significant CPP as compared to vehicle treated controls, and (iii) the reduction in (ii) is to a level comparable to that produced by buprenorphine alone and not statistically significant from vehicle treated controls.

### Example 2 (Prophetic)

### Sample study design to evaluate opioid-induced euphoria in humans

The study design to evaluate oxycodone-induced euphoria in humans in the presence and absence of low amounts of buprenorphine may be adapted according to the FDA's Guidance For Industry, Abuse-Deterrent Opioids-Evaluation and Labeling, January 2013.

A category 3 clinical abuse potential study will be implemented to evaluate oxycodone-induced euphoria. The study design will be a randomized, double-blind, placebo-controlled and positive comparator controlled crossover study. The study will be conducted in an oxycodone-experienced abuser population. A pre-qualification phase to identify subjects who can distinguish active drug from placebo reproducibly as a common enrichment strategy will be used to improve the power of the study to distinguish difference between treatments. The following treatments will be included in the study:

### Placebo

Positive control (fixed dose of oxycodone only without buprenorphine)
Active treatment (fixed dose oxycodone+dose 1 of buprenorphine)
Active treatment (fixed dose of oxycodone+dose 2 of buprenorphine)
Active treatment (fixed dose oxycodone+dose 3 of buprenorphine)
Note: The ‘fixed dose of oxycodone’ will be selected from the pre-qualification phase oxycodone-experienced abuser population.

Pharmacokinetics and pharmacodynamics assessments will be included in the study after each treatment for up to 24 hour post dose.

Primary pharmacodynamics measures to evaluate euphoria will be:
(1) Visual analog scales (VAS) for “At this Moment” Drug Liking using a bipolar scale, and
(2) Visual analog scales (VAS) for “High” using a unipolar scale
Secondary pharmacodynamics measures:

### (1) VAS for “Overall Drug Liking”

### (2) VAS for “Take Drug Again”

Additional assessments of interest include but not limited to:
Subject-rated assessments including ‘Overall Drug Liking’ and “Alertness”

### Data Interpretation

The primary analysis will be the difference in means of the Eₘₐₓ of “At this Moment” Drug Liking and Eₘₐₓ of “High”, where Eₘₐₓ is maximum pharmacodynamic response.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

1. A method of preventing or attenuating euphoria induced by an opioid other than buprenorphine comprising administering to a patient in need thereof an effective amount of buprenorphine to prevent or attenuate the euphoria induced by the administration of the other opioid.

2. The method of claim 1, wherein the other opioid is administered in an effective amount to provide an analgesic effect.

3. The method of claim 1, wherein the patient is administered the buprenorphine concurrently with the other opioid.

4. The method of claim 3, wherein the administration of the other opioid is initiated prior to administration of the buprenorphine.

5. The method of claim 4, wherein the patient initiates administration of the other opioid on a chronic basis prior to administration of the buprenorphine.

6. The method of claim 5, wherein the patient exhibits the opioid-induced euphoria prior to administration of the buprenorphine.

7. The method of claim 6, wherein the administration of the buprenorphine attenuates the opioid-induced euphoria induced by the other opioid.

8. The method of claim 1, wherein the administration of the other opioid is initiated concurrently with the administration of the buprenorphine.

9. The method of claim 8, wherein the patient is opioid naive.

10. The method of claim 8, wherein the patient is administered the other opioid on a chronic basis.

11. The method of claim 1, wherein the administration of the buprenorphine is initiated prior to initiating the administration of the other opioid.

12. The method of claim 1, wherein the other opioid has an Eₘₐₓ of greater than about 25%.

13. The method of claim 1, wherein the buprenorphine is selected from the group consisting of buprenorphine base, pharmaceutically acceptable salts thereof, solvates thereof, polymorphs thereof, and mixtures thereof.

14. The method of claim 13, wherein the buprenorphine is buprenorphine base, buprenorphine hydrochloride or buprenorphine levulinate.

15. The method of claim 1, wherein the buprenorphine is administered transdermally.

16-20. (canceled)

21. The method of claim 1, wherein the buprenorphine is administered by the same route as the other opioid.

22. The method of claim 21, wherein the other opioid and the buprenorphine are each administered by a route selected from the group consisting of oral, transdermal, sublingual, buccal, gingival, rectal, subcutaneous, intramuscular, intravenous and parenteral.

23. The method of claim 22, wherein the other opioid and the buprenorphine are both administered orally.

24. The method of claim 23, wherein the other opioid and the buprenorphine are administered orally in two separate dosage forms.

25. The method of claim 23, wherein the other opioid and the buprenorphine are administered together orally in a single dosage form.

26. The method of claim 25, wherein the single dosage form is a solid oral dosage form.

27. The method of claim 26, wherein the solid oral dosage form is a tablet.

28. The method of claim 26, wherein the solid oral dosage form is a capsule.

29. The method of claim 26, wherein the other opioid and the buprenorphine are both formulated for controlled release.

30. The method of claim 26, wherein the other opioid and the buprenorphine are both formulated for immediate release.

31. The method of claim 26, wherein the other opioid is formulated for controlled release and the buprenorphine is formulated for immediate release.

32. The method of claim 26, wherein the other opioid is formulated for immediate release and the buprenorphine is formulated for controlled release.

33. The method of claim 2, wherein the buprenorphine is administered by a different route than the other opioid.

34. The method of claim 33, wherein the buprenorphine and the other opioid are administered by different routes independently selected from the group consisting of oral, transdermal, sublingual, buccal, gingival, rectal, subcutaneous, intramuscular, intravenous and parenteral.

35-46. (canceled)

47. The method of claim 1, wherein the other opioid is selected from the group consisting of oxycodone, methadone, morphine, codeine, oxymorphone, fentanyl, hydrocodone, hydromorphone, tramadol and the pharmaceutically acceptable salts thereof.

48-66. (canceled)

67. The method of claim 2, wherein the buprenorphine is administered in an amount that does not cause a decrease in the analgesic effectiveness of the other opioid.

68. The method of claim 2, wherein the buprenorphine is administered in an amount that does not cause a substantial decrease in the analgesic effectiveness of the other opioid.

69. The method of claim 2, wherein the buprenorphine is administered in an amount that provides an increase in analgesia over the analgesia provided by the other opioid alone.

70. The method of claim 1, wherein the other opioid is administered in an effective amount to treat pain, diarrhea, cough or anxiety.

71-78. (canceled)

79. The method of claim 2, wherein the concentration of buprenorphine that negatively affects the analgesic effectiveness of the concurrently administered opioid is about 90 times, about 80 times, about 70 times, about 60 times, about 50 times, about 40 times, about 30 times, about 20 times, about 10 times, about 5 times, or about 2 times the concentration of buprenorphine that prevents or attenuates opioid-induced euphoria.

80. A pharmaceutical unit dosage form comprising an effective amount of buprenorphine to prevent or decrease euphoria induced by another opioid, and a therapeutically effective amount of the other opioid.

81-83. (canceled)

84. A kit comprising (i) a unit dose of an effective amount of buprenorphine to prevent or attenuate opioid-induced euphoria induced by another opioid and (ii) a unit dose of the other opioid in an effective amount to treat pain, diarrhea, cough or anxiety.

85-94. (canceled)
